(19) United States
(12) Patent Application Publication
(10) Pub. No.: US 2022/0000335 A1
(43) Pub. Date: Jan. 6, 2022

(54) VIDEO PROCESSOR, IMAGE PROCESSING METHOD, ENDOSCOPE, AND ENDOSCOPE SYSTEM

(71) Applicant: OLYMPUS CORPORATION, Tokyo (JP)

(72) Inventors: Erika YANAGIHARA, Tokyo (JP); Shinsuke TANI, Tokyo (JP)

(73) Assignee: OLYMPUS CORPORATION, Tokyo (JP)

(21) Appl. No.: 17/379,275

(22) Filed: Jul. 19, 2021

Related U.S. Application Data
(63) Continuation of application No. PCT/JP2019/001909, filed on Jan. 22, 2019.

(51) Int. Cl. A61B 1/00; A61B 1/06

(52) U.S. Cl. CPC A61B 1/00006; A61B 1/00016; A61B 1/00036; A61B 1/0661; A61B 1/00009; A61B 1/0005

(57) ABSTRACT

An endoscope system includes an endoscope, a video processor, and a parameter control device. The parameter control device causes the endoscope and the video processor to execute predetermined processing by controlling a plurality of parameters used in the endoscope and the video processor. The parameter control device includes a data collection unit, a determination unit, and a parameter determination unit. The determination unit determines contents of constraint processing by determining a plurality of pieces of information acquired by the data collection unit, and determines contents of recovery processing so that a function for displaying an endoscope image is recovered, the function being degraded through the constraint processing. The parameter determination unit determines one or more parameters used in the constraint processing and one or more parameters used in the recovery processing.

### CROSS REFERENCE TO RELATED APPLICATION

This application is a continuation application of PCT/JP2019/001909 filed on Jan. 22, 2019, the entire contents of which are incorporated herein by this reference.

### BACKGROUND OF THE INVENTION

### 1. Field of the Invention

The present invention relates to a video processor, an image processing method, an endoscope, and an endoscope system that are capable of executing constraint processing that selectively constrains operation of the endoscope.

### 2. Description of the Related Art

Recently, an endoscope device has been widely used in medical and industrial fields. In particular, an endoscope used in the medical field has been widely used for observation of an organ in a body cavity, medical treatment using a treatment instrument, a surgical operation under endoscope observation, and the like.

Recently, practical use of a battery-driven wireless endoscope on which a rechargeable battery is mounted has been started along with progress of semiconductor technologies and electric power consumption reduction due to use of an LED as an illumination light source. The wireless endoscope includes a wireless communication unit configured to perform wireless communication with a video processor, and compresses image data obtained through image pickup by an image pickup device and wirelessly transmits the compressed image data.

The wireless endoscope desirably can execute, as necessary, electric power consumption reducing processing that reduces an electric power consumption of the endoscope to prevent function decrease such as battery degradation by reducing internal temperature rise and to increase an operational time by reducing a consumption amount of the battery. In addition, to prevent wireless communication blackout, the wireless endoscope can desirably execute processing that changes a compression ratio of image data by, for example, increasing the compression ratio in a situation in which wireless environment is degraded, and decreasing the compression ratio to obtain an endoscope image of high image quality in an important scene.

WO 2017/029839 discloses a wireless endoscope configured to perform power saving operation that increases an image compression ratio and decreases an illumination light amount at battery replacement. Japanese Patent No. 4800695 discloses an endoscope device configured to reduce electric power consumption by controlling operation of each component of a body part of an endoscope device in accordance with internal temperature of the body part and an actual examination situation. WO 2016/052175 discloses a portable endoscope system configured to calculate a compression ratio of an endoscope image based on a result of determination of a procedure scene type.

### SUMMARY OF THE INVENTION

A video processor according to an aspect of the present invention is a video processor including a processor. The processor is configured to: acquire at least one piece of information of information related to temperature of a grasping portion of an endoscope, information related to wireless environment of wireless communication that transmits and receives image data obtained through image pickup by the endoscope, or information related to a remaining amount of a battery of the endoscope; and control a plurality of parameters. The processor determines, based on the at least one piece of information, contents of constraint processing that selectively constrains operation of the endoscope and contents of recovery processing that recovers a function for displaying an endoscope image, the function being degraded through the constraint processing; and determines a parameter for the constraint processing and a parameter used in the recovery processing.

An image processing method according to an aspect of the present invention is an image processing method of generating an endoscope image from image data acquired by an image pickup device of an endoscope. The image processing method includes: acquiring at least one piece of information of information related to temperature of a grasping portion of the endoscope, information related to wireless environment of wireless communication that transmits and receives the image data, or information related to a remaining amount of a battery of the endoscope; determining, based on the at least one piece of information, contents of constraint processing that selectively constrains operation of the endoscope and contents of recovery processing that recovers a function for displaying the endoscope image, the function being degraded through the constraint processing; and determining a parameter for the constraint processing and a parameter used in the recovery processing.

An endoscope according to an aspect of the present invention is an endoscope including a processor. The processor is configured to: acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and control a plurality of parameters. The processor determines, based on the at least one piece of information, contents of constraint processing that selectively constrains operation of the endoscope and contents of recovery processing that recovers a function for displaying an endoscope image, the function being degraded through the constraint processing; and determines a parameter for the constraint processing and a parameter used in the recovery processing.

An endoscope system according to an aspect of the present invention includes an endoscope, a video processor, and a processor. The processor is configured to: acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and control a plurality of parameters. The processor determines, based on the at least one piece of information, contents of constraint processing that selectively constrains operation of the endoscope and contents of recovery processing that recovers a function for displaying an endoscope image, the function being degraded through the constraint processing; and determines a parameter for the constraint processing and a parameter used in the recovery processing. The processor is provided in the endoscope.

An endoscope system according to another aspect of the present invention includes an endoscope, a video processor, and a processor. The processor is configured to: acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and control a plurality of parameters. The processor determines, based on the at least one piece of information, contents of constraint processing that selectively constrains operation of the endoscope and contents of recovery processing that recovers a function for displaying an endoscope image, the function being degraded through the constraint processing; and determines a parameter for the constraint processing and a parameter used in the recovery processing. The processor is provided in the video processor.

### BRIEF DESCRIPTION OF THE DRAWINGS

FIG. 1 is an explanatory diagram illustrating an entire configuration of an endoscope system according to a first embodiment of the present invention;

FIG. 2 is a functional block diagram illustrating configurations of an endoscope and a parameter control device of the endoscope system according to the first embodiment of the present invention;

FIG. 3 is a functional block diagram illustrating configurations of a video processor and a display unit of the endoscope system according to the first embodiment of the present invention;

FIG. 4 is an explanatory diagram illustrating an example of a hardware configuration of the endoscope system according to the first embodiment of the present invention;

FIG. 5 is a flowchart illustrating part of operation of the endoscope system according to the first embodiment of the present invention;

FIG. 6 is a flowchart illustrating another part of the operation of the endoscope system according to the first embodiment of the present invention;

FIG. 7 is a flowchart illustrating another part of the operation of the endoscope system according to the first embodiment of the present invention;

FIG. 8 is a functional block diagram illustrating configurations of an endoscope and a first part of a parameter control device in an endoscope system according to a second embodiment of the present invention; and

FIG. 9 is a functional block diagram illustrating configurations of a video processor and a second part of the parameter control device in the endoscope system according to the second embodiment of the present invention.

### DETAILED DESCRIPTION OF THE PREFERRED EMBODIMENTS

Embodiments of the present invention will be described below with reference to the accompanying drawings.

### First Embodiment

(Configuration of Endoscope System)

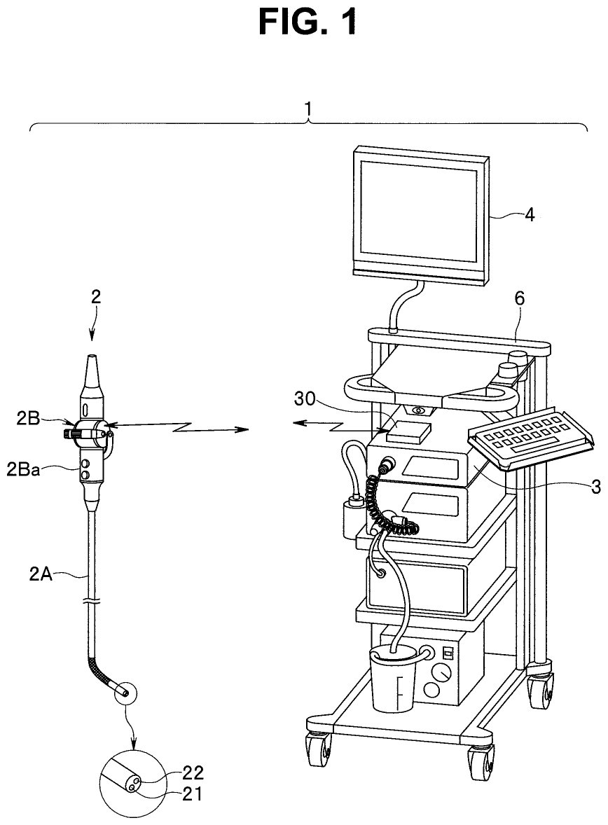

First, a schematic configuration of an endoscope system according to a first embodiment of the present invention will be described below. FIG. 1 is an explanatory diagram illustrating an entire configuration of an endoscope system 1 according to the present embodiment. The endoscope system 1 according to the present embodiment is a wireless endoscope system including a wireless endoscope 2 that is a battery-driven portable endoscope. Hereinafter, the wireless endoscope 2 is simply referred to as the endoscope 2.

The endoscope system 1 has a function for displaying an endoscope image obtained through image pickup by the endoscope 2. Specifically, the endoscope system 1 further includes a video processor 3 physically separated from the endoscope 2, and a display unit 4 connected to the video processor 3. The video processor 3 is wirelessly connected to the endoscope 2 and generates an endoscope image by performing predetermined image processing to be described later. The display unit 4 is configured of a monitor device or the like and displays the endoscope image and the like.

As illustrated in FIG. 1, the video processor 3, the display unit 4, and various medical instruments are placed on a cart 6 in an operation room. Examples of medical instruments placed on the cart 6 include devices such as an electrocautery scalpel device, a pneumoperitoneum apparatus, and a video recorder, and a gas cylinder filled with carbon dioxide.

Note that a configuration of the video processor 3 and the display unit 4 is not limited to an example illustrated in FIG. 1. For example, the endoscope system 1 may include a video processor integrated with a display unit in place of the video processor 3 and the display unit 4.

The endoscope 2 includes an elongated insertion portion 2A that is inserted into a body cavity, and an operation portion 2B including a grasping portion 2Ba that is grasped by a user. The operation portion 2B is provided at a proximal end portion of the insertion portion 2A.

The endoscope 2 further includes an image pickup unit 21 configured to generate image data through image pickup of an object, and an illumination unit 22 configured to illuminate the object. The object is a site such as an affected part in a subject. The image pickup unit 21 includes a non-illustrated image pickup device such as a CCD or a CMOS provided at a distal end portion of the insertion portion 2A.

The illumination unit 22 includes an illumination light source including a non-illustrated light-emitting element such as a light-emitting diode, and a non-illustrated lens provided at a distal end of the insertion portion 2A. Illumination light generated by the illumination light source is applied to the object through the lens. Return light of the illumination light from the object is imaged on an image pickup surface of the image pickup device of the image pickup unit 21. Note that the illumination light source may be provided in the operation portion 2B. In this case, the illumination light generated by the illumination light source is guided to the distal end of the insertion portion 2A through a non-illustrated light guide.

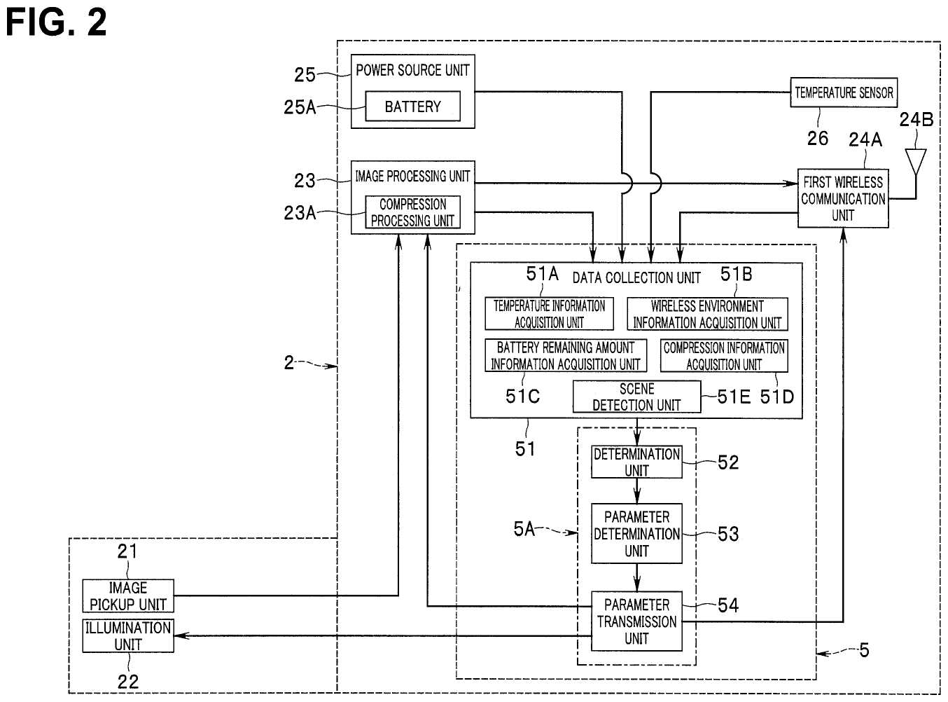

The endoscope system 1 further includes a parameter control device 5 according to the present embodiment. Note that the parameter control device 5 is illustrated in FIG. 2 to be described later. The parameter control device 5 is a device that causes the endoscope 2 and the video processor 3 to execute predetermined processing by controlling a plurality of parameters used by the endoscope 2 and the video processor 3.

(Configurations of Endoscope and Parameter Control Device)

Subsequently, configurations of the endoscope 2 and the parameter control device 5 will be described below in detail with reference to FIG. 2. FIG. 2 is a functional block diagram illustrating the configurations of the endoscope 2 and the parameter control device 5. In the present embodiment, the entire parameter control device 5 is provided in the endoscope 2.

As illustrated in FIG. 2, the endoscope 2 includes a first image processing unit (hereinafter simply referred to as an image processing unit) 23, a first wireless communication unit 24A, an antenna 24B, a power source unit 25, and a temperature sensor 26 in addition to the grasping portion 2B a, the image pickup unit 21, and the illumination unit 22. The image pickup unit 21 generates image data based on an object optical image through photoelectric conversion and outputs the image data to the image processing unit 23.

The image processing unit 23 includes a compression processing unit 23A. The compression processing unit 23A performs compression processing that generates compressed data by compressing the image data generated by the image pickup unit 21. In the compression processing, a compression parameter that defines a data amount of the compressed data is used. The compression parameter has a compression ratio and a correspondence relation of the compressed data. The image processing unit 23 outputs the generated compressed data to the first wireless communication unit 24A and outputs the present compression parameter to the parameter control device 5. In addition, the image processing unit 23 outputs the image data for detecting an endoscope scene as information related to the endoscope scene to the parameter control device 5.

The first wireless communication unit 24A includes a non-illustrated wireless transmission circuit configured to generate a wirelessly transmitted signal, and a non-illustrated wireless reception circuit configured to demodulate a wirelessly received signal. The first wireless communication unit 24A wirelessly transmits and receives a predetermined signal to and from the video processor 3 through the antenna 24B. The predetermined signal includes compressed data and a plurality of parameters to be described later.

The first wireless communication unit 24A further includes a non-illustrated environment detection circuit configured to detect a state of wireless communication environment (hereinafter simply referred to as wireless environment). The environment detection circuit detects, as the state of the wireless environment, for example, a wireless communication instrument existing in surroundings and using the same frequency band. The first wireless communication unit 24A outputs information related to the wireless environment detected by the environment detection circuit to the parameter control device 5. Note that the first wireless communication unit 24A may directly output a result of the detection by the environment detection circuit, or may calculate a forwardable data amount based on the result of the detection by the environment detection circuit and may output the calculated forwardable data amount. The forwardable data amount in wireless communication is defined in specifications of the wireless communication or changed depending on the wireless environment. The forwardable data amount is defined as, for example, a data amount that can be forwarded during a time in which image data of one frame is transmitted. The forwardable data amount decreases, for example, as the number of wireless communication instruments using the same frequency band increases.

Note that the first wireless communication unit 24A and a second wireless communication unit to be described later may be able to perform wireless communication by using a plurality of bands such as a 60-GHz band and a 5-GHz band. In this case, the 60-GHz band is used to, for example, transmit and receive compressed data. The 5-GHz band is used to, for example, transmit and receive a plurality of parameters.

The power source unit 25 includes a battery 25A and supplies electric power of the battery 25A to each component of the endoscope 2 including the image pickup unit 21, the illumination unit 22, the image processing unit 23, and the first wireless communication unit 24A. The battery 25A is mountable on, for example, the operation portion 2B (refer to FIG. 1). In addition, the power source unit 25 includes a non-illustrated battery remaining amount detection circuit configured to detect a remaining amount of the battery 25A. The power source unit 25 outputs information of the detected remaining amount of the battery 25A to the parameter control device 5.

The temperature sensor 26 is able to measure temperature of the grasping portion 2Ba (refer to FIG. 1), and outputs a measurement result of the temperature of the grasping portion 2Ba to the parameter control device 5. Note that the endoscope 2 may include, in addition to the temperature sensor 26, one or more temperature sensors configured to measure temperature of each component of the endoscope 2 except for the grasping portion 2Ba and the temperature sensor 26.

As illustrated in FIG. 2, the parameter control device 5 includes a data collection unit 51, a determination unit 52, a parameter determination unit 53, and a parameter transmission unit 54. The determination unit 52, the parameter determination unit 53, and the parameter transmission unit 54 are included in a control unit 5A as a main part of the parameter control device 5. In other words, the determination unit 52 and the parameter determination unit 53 are provided in the endoscope 2. The data collection unit 51 acquires a plurality of pieces of information related to the endoscope system 1. A configuration of the data collection unit 51 will be described later.

Processing that selectively constrains operation of the endoscope 2 is referred to as constraint processing. In addition, processing that operates the video processor 3 to recover a function for displaying an endoscope image, which is degraded through the constraint processing is referred to as recovery processing. The determination unit 52 determines contents of the constraint processing and contents of the recovery processing by determining the plurality of pieces of information acquired by the data collection unit 51.

Specifically, the function for displaying an endoscope image is a function for causing the display unit 4 to continuously display an endoscope image that satisfies needs of the user. In the present embodiment, the function for displaying an endoscope image includes at least a battery operation function that operates the endoscope 2 by the battery 25A, a wireless transmission function that wirelessly transmits image data from the endoscope 2 to the video processor 3, and an image quality maintaining function that maintains image quality of the endoscope image at a predetermined level or higher. When the constraint processing is executed, the battery operation function and the wireless transmission function are maintained but the image quality maintaining function is degraded. In contrast, in the present embodiment, the image quality maintaining function is recovered by executing the recovery processing.

The parameter determination unit 53 determines one or more parameters used in the constraint processing having contents determined by the determination unit 52, and one or more parameters used in the recovery processing having contents determined by the determination unit 52.

The parameter transmission unit 54 transmits the plurality of parameters determined by the parameter determination unit 53 to each component of the endoscope 2 and the video processor 3. In the endoscope 2, the illumination unit 22 and the compression processing unit 23A receive the parameters transmitted from the parameter transmission unit 54. In the video processor 3, a main control unit to be described later receives the parameters transmitted from the parameter transmission unit 54.

The endoscope 2 further includes a non-illustrated main control unit. The main control unit controls each component of the endoscope 2 including the parameter control device 5, and also controls the power source unit 25 to supply power to each component of the endoscope 2 including the parameter control device 5.

(Configuration of Video Processor)

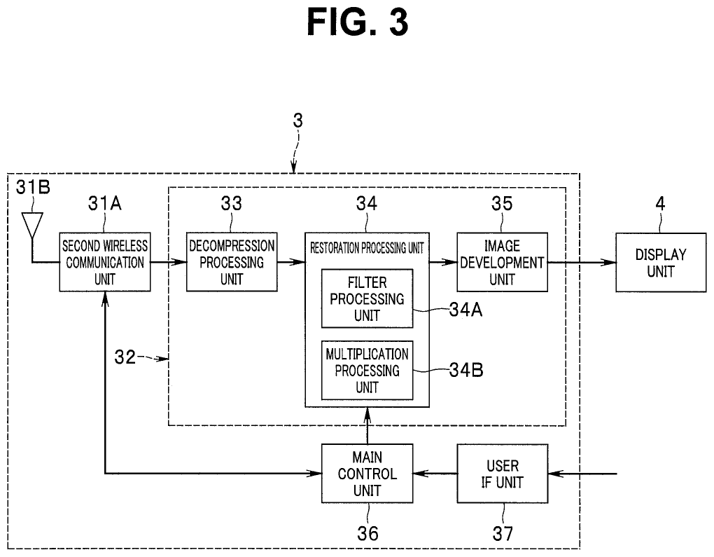

Subsequently, a configuration of the video processor 3 will be described below with reference to FIG. 3. FIG. 3 is a functional block diagram illustrating a configuration of the video processor 3 and the display unit 4. As illustrated in FIG. 3, the video processor 3 includes a second wireless communication unit 31A, an antenna 31B, a second image processing unit (hereinafter simply referred to as an image processing unit) 32, a main control unit 36, and a user interface unit (hereinafter referred to as a user IF unit) 37.

The second wireless communication unit 31A and the antenna 31B may be built in a main body of the video processor 3 or may be built in a wireless receiver 30 separated from the main body of the video processor 3. FIG. 1 illustrates the wireless receiver 30. The wireless receiver 30 is connected to the main body of the video processor 3 through a non-illustrated connector.

The second wireless communication unit 31A includes a non-illustrated wireless transmission circuit configured to generate a wirelessly transmitted signal, and a non-illustrated wireless reception circuit configured to demodulate a wirelessly received signal. The second wireless communication unit 31A wirelessly transmits and receives a predetermined signal to and from the endoscope 2 through the antenna 31B. The predetermined signal includes the compressed data transmitted by the first wireless communication unit 24A and the plurality of parameters transmitted by the parameter transmission unit 54. The second wireless communication unit 31A outputs the compressed data to the image processing unit 32, and outputs the plurality of parameters to the main control unit 36.

The second wireless communication unit 31A may further include a non-illustrated environment detection circuit configured to detect the state of the wireless environment. Functions of the environment detection circuit of the second wireless communication unit 31A are the same as functions of the environment detection circuit of the first wireless communication unit 24A. The second wireless communication unit 31A outputs information related to the wireless environment detected by the environment detection circuit to the parameter control device 5 through wireless communication between the endoscope 2 and the video processor 3. Contents of the information related to the wireless environment and outputted from the second wireless communication unit 31A are the same as contents of the information related to the wireless environment and outputted from the first wireless communication unit 24A described above.

The image processing unit 32 generates decompressed image data corresponding to image data by decompressing the compressed data, and generates an endoscope image by performing predetermined image processing on the decompressed image data. In the present embodiment, the image processing unit 32 includes a decompression processing unit 33 configured to generate the decompressed image data, a restoration processing unit 34, and an image development unit 35.

The restoration processing unit 34 performs at least one piece of image restoration processing on the decompressed image data to improve image quality of the endoscope image. In the present embodiment, in particular, the restoration processing unit 34 is able to perform, as the at least one piece of image restoration processing, brightness correction processing that corrects brightness of the decompressed image data. Specifically, the restoration processing unit 34 includes a filter processing unit 34A and a multiplication processing unit 34B that execute the brightness correction processing.

The filter processing unit 34A performs filter processing that corrects brightness of any one pixel of the decompressed image data by using a plurality of pixel values in a predetermined region including the one pixel and a plurality of pixels surrounding the one pixel, and a first brightness parameter. The filter processing may be, for example, processing that, for each channel of RGB, multiplies values of brightness of the plurality of surrounding pixels by coefficients (weights) and adds the multiplied values to a value of brightness of the one pixel. In this case, the first brightness parameter may be the coefficients (weights) by which the values of brightness of the plurality of pixels are multiplied.

The multiplication processing unit 34B performs multiplication processing that corrects brightness of any one pixel by using a pixel value of the one pixel and a second brightness parameter. The multiplication processing may be processing that multiplies a luminance value of the one pixel by the second brightness parameter as a multiplier. In this case, the second brightness parameter may be a constant or may be a value that changes in accordance with the luminance value as in gamma correction. In the latter case, the multiplication processing is performed by using a table indicating a relation between the luminance value and the second brightness parameter.

Note that, as an effect of the filter processing is stronger, the decompressed image data after correction is brighter but a resolution of the decompressed image data after correction is lower. In addition, as an effect of the multiplication processing is stronger, the decompressed image data after correction is brighter but noise of the decompressed image data after correction is larger. Thus, in order to obtain the endoscope image of high image quality and high resolution by performing the filter processing and the multiplication processing so that the endoscope image becomes brighter, it is needed to set the first brightness parameter to avoid excess decrease of the resolution of the decompressed image data after correction, and to set the second brightness parameter to avoid excess increase of the noise of the decompressed image data after correction.

The image development unit 35 performs image development processing that generates the endoscope image by converting the decompressed image data into a format displayable on the display unit 4. The image processing unit 32 outputs the generated endoscope image to the display unit 4.

The user IF unit 37 is an interface configured to receive a user operation. Specifically, the user IF unit 37 includes, for example, a front panel and various switches of a control system, and outputs an operation signal based on the user operation to the main control unit 36. Examples of the user operation include activation of the endoscope system 1, power-off of the endoscope system 1, specification of an observation mode of the endoscope 2, setting related to image display, and setting of an operation mode of the endoscope 2.

The main control unit 36 controls each component of the video processor 3 and also controls a non-illustrated power source unit provided in the video processor 3 to supply power to each component of the video processor 3. The main control unit 36 receives a parameter transmitted from the parameter transmission unit 54 and outputs the received parameter to the restoration processing unit 34. The main control unit 36 outputs information based on an operation signal inputted through the user IF unit 37 to each component of the video processor 3, and also outputs the information to the non-illustrated main control unit of the endoscope 2 through wireless communication between the endoscope 2 and the video processor 3. Accordingly, the main control unit 36 can provide various instructions to each component of the endoscope 2 and the video processor 3.

(Hardware Configuration)

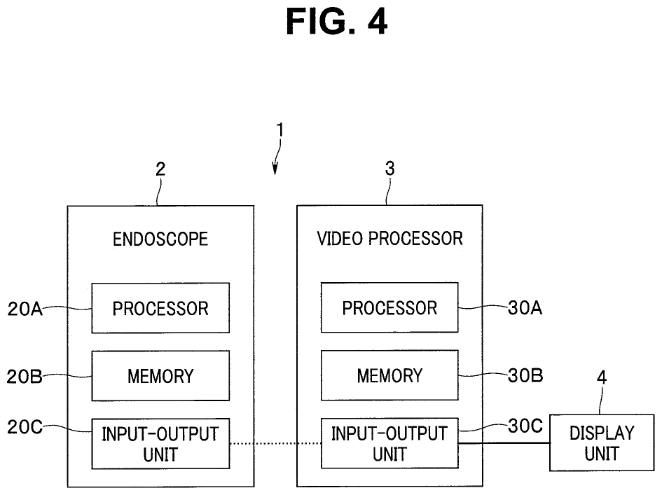

Subsequently, a hardware configuration of the endoscope system 1 will be described below with reference to FIG. 4. FIG. 4 is an explanatory diagram illustrating an example of the hardware configuration of the endoscope system 1. In the example illustrated in FIG. 4, the endoscope 2 includes a processor 20A, a memory 20B, and an input-output unit 20C. The video processor 3 includes a processor 30A, a memory 30B, and an input-output unit 30C.

The processor 20A is used to execute functions of the image processing unit 23, the first wireless communication unit 24A, the power source unit 25, the non-illustrated main control unit, and the like as components of the endoscope 2, and functions of the data collection unit 51, the determination unit 52, the parameter determination unit 53, and the parameter transmission unit 54 as components of the parameter control device 5. The processor 30A is used to execute functions of the second wireless communication unit 31A, the image processing unit 32, the main control unit 36, and the like as components of the video processor 3. The processors 20A and 30A are each configured of, for example, a field programmable gate array (FPGA). At least some of a plurality of components of the endoscope 2, the video processor 3, and the parameter control device 5 may be configured as circuit blocks in the FPGA.

The memories 20B and 30B are each configured of a rewritable storage element such as RAM. The input-output unit 20C is used to perform signal transmission and reception between the endoscope 2 and outside. The input-output unit 30C is used to perform signal transmission and reception between the video processor 3 and outside. In the present embodiment, in particular, wireless signal transmission and reception between the endoscope 2 and the video processor 3 are performed by using the input-output units 20C and 30C.

Note that the processors 20A and 30A may be each configured of a central processing unit (hereinafter referred to as a CPU). In this case, the functions of components of the endoscope 2 and the parameter control device 5 may be achieved as the CPU reads a program from the memory 20B or a non-illustrated storage device and executes the program. Similarly, the functions of components of the video processor 3 may be achieved as the CPU reads a program from the memory 30B or a non-illustrated storage device and executes the program.

The hardware configuration of the endoscope system 1 is not limited to the example illustrated in FIG. 4. For example, a plurality of components of the endoscope 2, the video processor 3, and the parameter control device 5 may be each configured as a separate electronic circuit.

(Operation of Parameter Control Device)

Subsequently, operation of the parameter control device 5 will be described below.

(Configuration and Operation of Data Collection Unit)

First, a configuration and operation of the data collection unit 51 will be described below with reference to FIG. 2. The data collection unit 51 acquires, as the plurality of pieces of information, at least one of information related to the temperature of the grasping portion 2Ba, information related to wireless environment between the first wireless communication unit 24A and the second wireless communication unit 31A, or information related to the remaining amount of the battery 25A, and also, information related to an endoscope scene. In the present embodiment, the data collection unit 51 includes a temperature information acquisition unit 51A, a wireless environment information acquisition unit 51B, a battery remaining amount information acquisition unit 51C, and a scene detection unit 51E. In other words, the temperature information acquisition unit 51A, the wireless environment information acquisition unit 51B, the battery remaining amount information acquisition unit 51C, and the scene detection unit 51E are provided in the endoscope 2.

The temperature information acquisition unit 51A acquires the information related to the temperature of the grasping portion 2Ba. In the present embodiment, the temperature information acquisition unit 51A receives the measurement result of the temperature of the grasping portion 2Ba, which is outputted from the temperature sensor 26.

The wireless environment information acquisition unit 51B acquires the information related to the wireless environment. In the present embodiment, the wireless environment information acquisition unit 51B receives, the information related to the wireless environment, which is outputted from the first wireless communication unit 24A. The wireless environment information acquisition unit 51B acquires, as the information related to the wireless environment, the result of the detection by the environment detection circuit of the first wireless communication unit 24A or the forwardable data amount calculated based on the result of the detection by the environment detection circuit. When the wireless environment information acquisition unit 51B acquires the result of the detection by the environment detection circuit, the wireless environment information acquisition unit 51B may calculate the forwardable data amount based on the result of the detection by the environment detection circuit.

Note that when the second wireless communication unit 31A includes an environment detection circuit as described above, the wireless environment information acquisition unit 51B may receive the information related to the wireless environment, which is outputted from the second wireless communication unit 31A. In this case, the information related to the wireless environment, which is acquired by the wireless environment information acquisition unit 51B may be information outputted from the first wireless communication unit 24A or may be information outputted from the second wireless communication unit 31A.

The battery remaining amount information acquisition unit 51C acquires the information related to the remaining amount of the battery 25A. In the present embodiment, the battery remaining amount information acquisition unit 51C receives the information related to the remaining amount of the battery 25A, which is outputted from the power source unit 25.

The scene detection unit 51E acquires information related to an endoscope scene. In the present embodiment, the scene detection unit 51E receives image data for detecting an endoscope scene as information related to the endoscope scene outputted from the image processing unit 23. The scene detection unit 51E detects an endoscope scene by analyzing acquired image data. Examples of the endoscope scene include a detailed-check scene corresponding to a case of detailed-check observation of a blood vessel or the like, a screening scene corresponding to, for example, a case of search for an anomalous part while moving the insertion portion 2A, and an external scene corresponding to a case of external positioning of the insertion portion 2A.

The data collection unit 51 further includes a compression information acquisition unit 51D. The compression information acquisition unit 51D acquires information related to the compression processing. In the present embodiment, the compression information acquisition unit 51D receives the compression parameter outputted from the image processing unit 23.

(Operation of Determination Unit)

Subsequently, operation of the control unit 5A of the parameter control device 5, in other words, operation of the determination unit 52, the parameter determination unit 53, and the parameter transmission unit 54 will be described below with reference to FIGS. 2 and 3. First, the operation of the determination unit 52 will be described below. The determination unit 52 determines, as a plurality of pieces of information, the information related to the temperature of the grasping portion 2Ba, which is acquired by the temperature information acquisition unit 51A, the information related to the wireless environment, which is acquired by the wireless environment information acquisition unit 51B, the information related to the remaining amount of the battery 25A, which is acquired by the battery remaining amount information acquisition unit 51C, and the information related to the endoscope scene, which is acquired by the scene detection unit 51E.

The operation of the determination unit 52 for the information related to the temperature of the grasping portion 2Ba, the information related to the remaining amount of the battery 25A, and the information related to the endoscope scene is as follows. The determination unit 52 determines whether the temperature of the grasping portion 2Ba is equal to or higher than a predetermined temperature threshold value, and determines whether the remaining amount of the battery 25A is smaller than a predetermined battery threshold value. When the determination unit 52 determines at least one of a condition that the temperature of the grasping portion 2Ba is equal to or higher than the predetermined temperature threshold value or a condition that the remaining amount of the battery 25A is smaller than the predetermined battery threshold value, the determination unit 52 determines to execute electric power consumption reducing processing as the constraint processing. Hereinafter, the condition that the temperature of the grasping portion 2Ba is equal to or higher than the predetermined temperature threshold value and the condition that the remaining amount of the battery 25A is smaller than the predetermined battery threshold value are referred to as an execution condition of the electric power consumption reducing processing.

The electric power consumption reducing processing is processing that operates the endoscope 2 so that consumption of electric power of the battery 25A is smaller than when the electric power consumption reducing processing is not executed. In the present embodiment, the electric power consumption reducing processing includes at least illumination light amount change processing among the illumination light amount change processing and compression amount change processing, the illumination light amount change processing being processing that changes an illumination light amount of the illumination unit 22, the compression amount change processing being processing that changes the data amount of the compressed data. In the present embodiment, determination of whether to execute only the illumination light amount change processing or execute both the illumination light amount change processing and the compression amount change processing is performed by using a result of the determination of the information related to the remaining amount of the battery 25A and the information related to the endoscope scene as described later.

When the determination unit 52 determines to execute the electric power consumption reducing processing, the determination unit 52 determines, as the recovery processing, to change contents of the brightness correction processing performed by the restoration processing unit 34. In the present embodiment, the determination unit 52 determines to change contents of the filter processing performed by the filter processing unit 34A and contents of the multiplication processing performed by the multiplication processing unit 34B. Specifically, the determination unit 52 determines to execute strong filter processing that is the filter processing having an effect stronger than when the recovery processing is not executed, and determines to execute strong multiplication processing that is the multiplication processing having an effect stronger than when the recovery processing is not executed.

The determination unit 52 also determines whether the endoscope scene is a scene (hereinafter referred to as a high-resolution scene) that needs the endoscope image of high resolution, such as the detailed-check scene. The determination unit 52 changes contents of the electric power consumption reducing processing and contents of the brightness correction processing, depending on whether the endoscope scene is the high-resolution scene.

Specifically, the determination unit 52 determines to preferentially execute the illumination light amount change processing among the illumination light amount change processing and the compression amount change processing when the determination unit 52 determines that the execution condition of the electric power consumption reducing processing is satisfied and that the endoscope scene is the high-resolution scene. In this case, the determination unit 52 may determine to execute only the illumination light amount change processing. Alternatively, the determination unit 52 may determine to execute both the illumination light amount change processing and the compression amount change processing so that an amount of reduction of electric power consumption of the battery 25A through the illumination light amount change processing is larger than an amount of reduction of electric power consumption of the battery 25A through the compression amount change processing.

Note that when the determination unit 52 determines that the execution condition of the electric power consumption reducing processing is satisfied but not that the endoscope scene is the high-resolution scene, the determination unit 52 determines to execute both the illumination light amount change processing and the compression amount change processing, and determines to execute the compression amount change processing so that the data amount of the compressed data is smaller than when the determination unit 52 determines that the endoscope scene is the high-resolution scene.

When the determination unit 52 determines to execute the electric power consumption reducing processing and determines that the endoscope scene is the high-resolution scene, the determination unit 52 determines to execute the strong filter processing in which the effect of the filter processing is stronger than when it is not determined that the endoscope scene is the high-resolution scene. Note that the determination unit 52 determines to execute the strong multiplication processing described above irrespective of whether the determination unit 52 determines that the endoscope scene is the high-resolution scene.

The operation of the determination unit 52 for the information related to the wireless environment and the information related to the endoscope scene is as follows. In the present embodiment, the determination unit 52 determines whether the wireless environment is degraded by determining whether the forwardable data amount is smaller than a predetermined threshold value. Note that when the wireless environment information acquisition unit 51B acquires or calculates the forwardable data amount, the determination unit 52 uses the forwardable data amount acquired or calculated by the wireless environment information acquisition unit 51B. When the wireless environment information acquisition unit 51B acquires the result of the detection by the environment detection circuit but does not calculate the forwardable data amount, the determination unit 52 calculates the forwardable data amount by using the result of the detection by the environment detection circuit, which is acquired by the wireless environment information acquisition unit 51B.

When the determination unit 52 determines that the forwardable data amount is smaller than the predetermined threshold value, the determination unit 52 determines to execute wireless transmission amount reducing processing as the constraint processing. Hereinafter, a condition that the forwardable data amount is smaller than the predetermined threshold value is referred to as an execution condition of the wireless transmission amount reducing processing. The wireless transmission amount reducing processing is processing that operates the endoscope 2 so that an amount of wireless transmission between the endoscope 2 and the video processor 3 is smaller than when the wireless transmission amount reducing processing is not executed. In the present embodiment, the wireless transmission amount reducing processing includes the compression amount change processing that changes the data amount of the compressed data.

When the determination unit 52 determines to execute the wireless transmission amount reducing processing, the determination unit 52 determines, as the recovery processing, to change contents of the brightness correction processing performed by the restoration processing unit 34. In the present embodiment, the determination unit 52 determines to change contents of the filter processing performed by the filter processing unit 34A and contents of the multiplication processing performed by the multiplication processing unit 34B. Specifically, the determination unit 52 determines to execute weak filter processing that is the filter processing having an effect weaker than when the recovery processing is not executed, and determines to execute the strong multiplication processing described above.

The determination unit 52 changes contents of the wireless transmission amount reducing processing and contents of the brightness correction processing, depending on whether the endoscope scene is the high-resolution scene. Specifically, when the determination unit 52 determines that the execution condition of the wireless transmission amount reducing processing is satisfied and that the endoscope scene is the high-resolution scene, the determination unit 52 determines to execute the wireless transmission amount reducing processing so that the data amount of the compressed data is smaller than when the wireless transmission amount reducing processing is not executed but the data amount of the compressed data is larger than when it is not determined that the endoscope scene is the high-resolution scene.

When the determination unit 52 determines to execute the wireless transmission amount reducing processing and determines that the endoscope scene is the high-resolution scene, the determination unit 52 determines to execute the weak filter processing having an effect weaker than when the recovery processing is not executed but stronger than when it is not determined that the endoscope scene is the high-resolution scene, and determines to execute the strong multiplication processing having an effect stronger than when the recovery processing is not executed but weaker than when it is not determined that the endoscope scene is the high-resolution scene.

(Operation of Parameter Determination Unit)

Subsequently, the operation of the parameter determination unit 53 will be described below. The operation of the parameter determination unit 53 for the electric power consumption reducing processing is as follows. When the determination unit 52 determines to execute the illumination light amount change processing, the parameter determination unit 53 determines an illumination parameter so that the illumination light amount of the illumination unit 22 is smaller than when the electric power consumption reducing processing is not executed, the illumination parameter defining the illumination light amount. When the determination unit 52 determines to execute the compression amount change processing, the parameter determination unit 53 determines the compression parameter so that the data amount of the compressed data is smaller than when the electric power consumption reducing processing is not executed.

When the determination unit 52 determines that the endoscope scene is the high-resolution scene and determines to execute both the illumination light amount change processing and the compression amount change processing, the parameter determination unit 53 determines the illumination parameter and the compression parameter so that the amount of reduction of electric power consumption of the battery 25A through the illumination light amount change processing is larger than the amount of reduction of electric power consumption of the battery 25A through the compression amount change processing.

The operation of the parameter determination unit 53 for the recovery processing corresponding to the electric power consumption reducing processing is as follows. The parameter determination unit 53 determines a brightness parameter so that an effect of the brightness correction processing that brightens the endoscope image is stronger than when the recovery processing is not executed, the brightness parameter defining a relation between brightness of the decompressed image data before correction and brightness of the decompressed image data after correction. Note that when the determination unit 52 determines that the endoscope scene is the high-resolution scene, the parameter determination unit 53 determines the brightness parameter so that the effect of the brightness correction processing is stronger than when the recovery processing is not executed but the effect of the brightness correction processing is weaker than when it is not determined that the endoscope scene is the high-resolution scene.

In the present embodiment, the parameter determination unit 53 determines, as the brightness parameter, a first brightness parameter used in the filter processing and a second brightness parameter used in the multiplication processing. Specifically, the parameter determination unit 53 determines the first brightness parameter so that the effect of the filter processing is stronger than when the recovery processing is not executed, and determines the second brightness parameter so that the effect of the multiplication processing is stronger than when the recovery processing is not executed. Note that when the determination unit 52 determines that the endoscope scene is the high-resolution scene, the parameter determination unit 53 determines the first brightness parameter so that the effect of the filter processing is stronger than when it is not determined that the endoscope scene is the high-resolution scene.

The operation of the parameter determination unit 53 for the wireless transmission amount reducing processing is as follows. When the determination unit 52 determines to execute the wireless transmission amount reducing processing, the parameter determination unit 53 determines the compression parameter so that the data amount of the compressed data is smaller than when the wireless transmission amount reducing processing is not executed. Note that when the determination unit 52 determines that the endoscope scene is the high-resolution scene, the determination unit 52 determines the compression parameter so that the data amount of the compressed data is smaller than when the wireless transmission amount reducing processing is not executed but the data amount of the compressed data is larger than when it is not determined that the endoscope scene is the high-resolution scene.

The operation of the parameter determination unit 53 for the recovery processing corresponding to the wireless transmission amount reducing processing is as follows. The parameter determination unit 53 determines the first brightness parameter used in the filter processing so that the effect of the filter processing is weaker than when the recovery processing is not executed, and determines the second brightness parameter used in the multiplication processing so that the effect of the multiplication processing is stronger than when the recovery processing is not executed. Note that when the determination unit 52 determines that the endoscope scene is the high-resolution scene, the parameter determination unit 53 determines the first brightness parameter so that the effect of the filter processing is weaker than when the recovery processing is not executed but the effect of the filter processing is stronger than when it is not determined that the endoscope scene is the high-resolution scene, and determines the second brightness parameter so that the effect of the multiplication processing is stronger than when the recovery processing is not executed but the effect of the multiplication processing is weaker than when it is not determined that the endoscope scene is the high-resolution scene.

Note that in the present embodiment, the parameter determination unit 53 may receive the compression parameter acquired by the compression information acquisition unit 51D. In this case, the parameter determination unit 53 may determine the compression parameter used in next compression processing based on a result of the determination by the determination unit 52 and the compression parameter used in the compression processing right before.

(Operation of Parameter Transmission Unit)

Subsequently, the operation of the parameter transmission unit 54 will be described below. The parameter transmission unit 54 transmits the illumination parameter to the illumination unit 22, transmits the compression parameter to the compression processing unit 23A, and transmits the first and second brightness parameters to the main control unit 36 of the video processor 3. The illumination unit 22 changes the illumination light amount of the illumination unit 22 based on the received illumination parameter. The compression processing unit 23A performs the compression processing by using the received compression parameter.

The main control unit 36 outputs the received first brightness parameter to the filter processing unit 34A of the restoration processing unit 34, and outputs the received second brightness parameter to the multiplication processing unit 34B of the restoration processing unit 34. The filter processing unit 34A performs the filter processing by using the first brightness parameter. The multiplication processing unit 34B performs the multiplication processing by using the second brightness parameter.

(Standard Processing)

Processing that the parameter control device 5 causes the endoscope 2 and the video processor 3 to execute when the constraint processing is not executed, in other words, when the electric power consumption reducing processing and the wireless transmission amount reducing processing are not executed is referred to as standard processing. The determination unit 52 determines to execute the standard processing when the determination unit 52 does not determine that the execution condition of the electric power consumption reducing processing is satisfied nor determine that the execution condition of the wireless transmission amount reducing processing is satisfied. In this case, the determination unit 52 may determine contents of the standard processing by determining the information related to the endoscope scene, which is acquired by the scene detection unit 51E. The parameter determination unit 53 also determines the illumination parameter, the compression parameter, the first brightness parameter, and the second brightness parameter used in the standard processing having contents determined by the determination unit 52.

(A Series of Operations Related to Parameter Control Device)

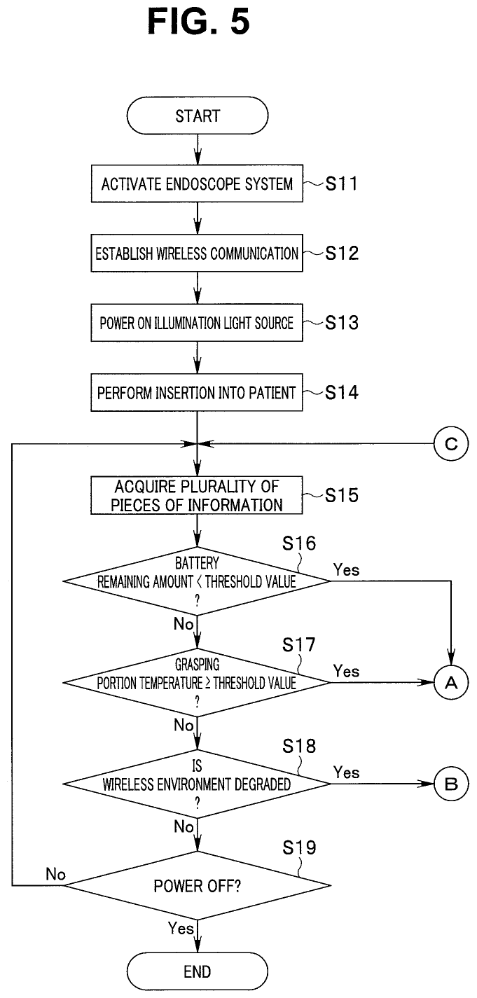

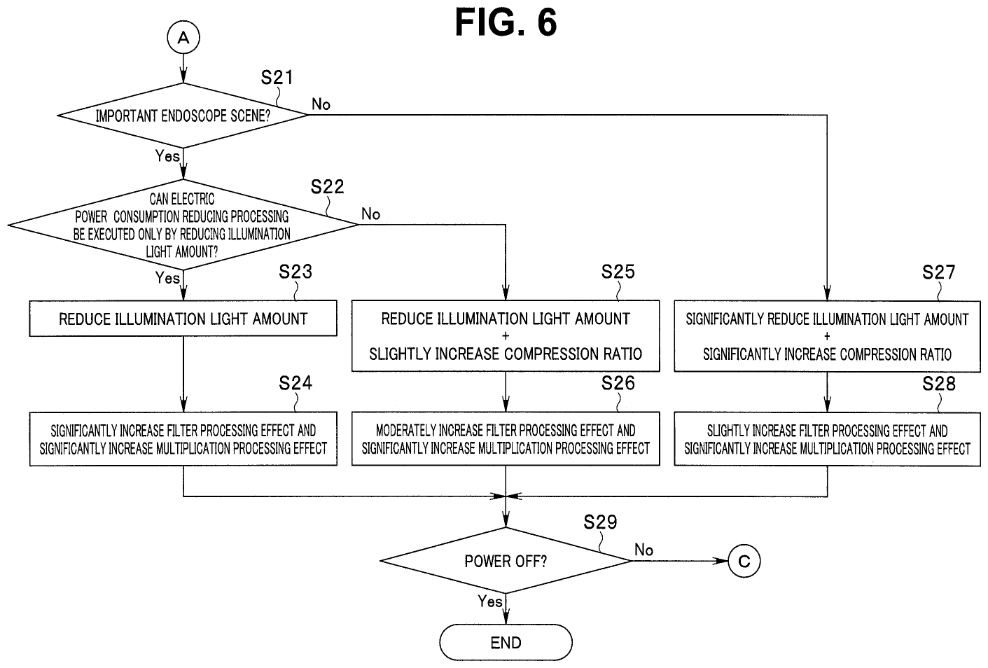

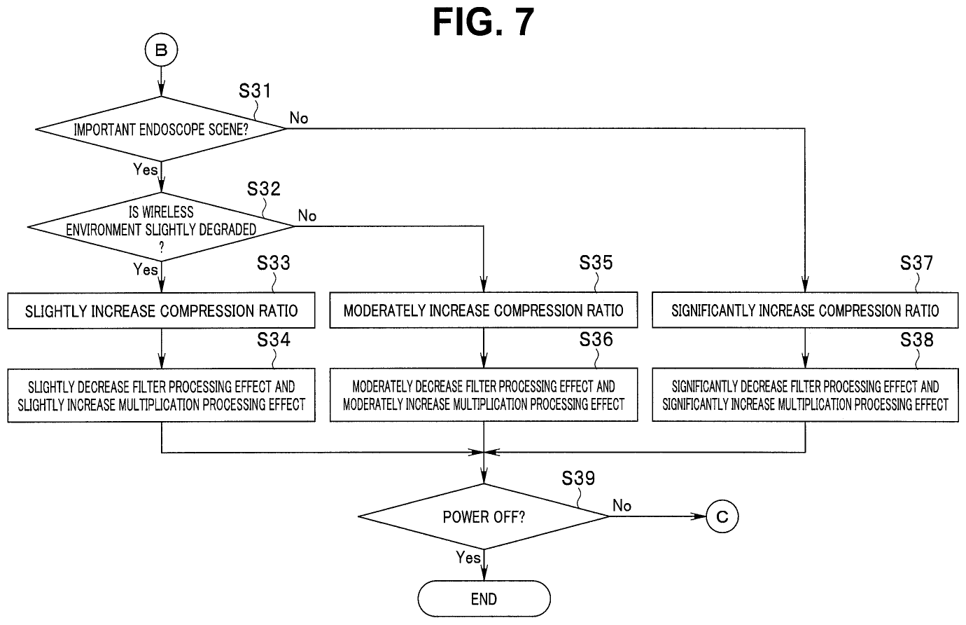

Subsequently, a specific example of a series of operations related to the parameter control device 5 in operation of the endoscope system 1 will be described below with reference to FIGS. 2, 3, and 5 to 7. FIG. 5 is a flowchart illustrating part of the operation of the endoscope system 1. FIG. 6 is a flowchart illustrating another part of the operation of the endoscope system 1. FIG. 7 is a flowchart illustrating another part of the operation of the endoscope system 1.

As illustrated in FIG. 5, first in the series of operations, an operation signal that activates the endoscope system 1 is inputted to the main control unit 36 through the user IF unit 37 as, for example, the user operates a switch or the like for activating the endoscope system 1. The main control unit 36 activates the endoscope system 1 based on the inputted operation signal (step S11). Subsequently, wireless communication connection is established between the endoscope 2 and the video processor 3 as the main control unit of the endoscope 2 controls the first wireless communication unit 24A and the main control unit 36 of the video processor 3 controls the second wireless communication unit 31A (step S12).

Subsequently, the illumination light source is powered on as the main control unit of the endoscope 2 controls the illumination unit 22 (step S13), and the endoscope 2 and the video processor 3 start execution of the standard processing. Subsequently, the user starts an insertion operation that inserts the insertion portion 2A of the endoscope 2 into a body of a patient (step S14).

Subsequently, the data collection unit 51 of the parameter control device 5 acquires a plurality of pieces of information related to the endoscope system 1 (step S15). Subsequently, the determination unit 52 of the parameter control device 5 determines the information related to the remaining amount of the battery 25A (step S16). When the determination unit 52 determines that the remaining amount of the battery 25A is smaller than the predetermined battery threshold value (Yes), step S21 in FIG. 6 is executed.

When the determination unit 52 determines that the remaining amount of the battery 25A is not smaller than the predetermined battery threshold value at step S16 (No), in other words, when the remaining amount of the battery 25A is equal to or larger than the predetermined battery threshold value, the determination unit 52 subsequently determines the information related to the temperature of the grasping portion 2Ba (step S17). When the determination unit 52 determines that the temperature of the grasping portion 2B a is equal to or higher than the predetermined temperature threshold value (Yes), step S21 in FIG. 6 is executed.

When the determination unit 52 determines that the temperature of the grasping portion 2Ba is not equal to nor larger than the predetermined temperature threshold value at step S17 (No), in other words, when the temperature of the grasping portion 2Ba is lower than the predetermined temperature threshold value, the determination unit 52 subsequently determines the information related to the wireless environment (step S18). At step S18, the determination unit 52 determines whether the wireless environment is degraded by determining whether the forwardable data amount is smaller than the predetermined threshold value. When the determination unit 52 determines that the forwardable data amount is smaller than the predetermined threshold value and the wireless environment is degraded (Yes), step S31 in FIG. 7 is executed.

At step S18, when the determination unit 52 determines that the forwardable data amount is equal to or larger than the predetermined threshold value and the wireless environment is not degraded (No), for example, the main control unit 36 subsequently determines whether to power off the endoscope system 1 (step S19). Specifically, the main control unit 36 determines whether an operation signal that powers off the endoscope system 1 is inputted. The operation signal is inputted to the main control unit 36 through the user IF unit 37, for example, as the user operates a switch or the like for powering off the endoscope system 1. When the operation signal is not inputted to the main control unit 36, the main control unit 36 determines not to power off the endoscope system 1 (No), and step S15 is executed again. When the operation signal is inputted to the main control unit 36, the main control unit 36 determines to power off the endoscope system 1 (Yes), and the series of operations are ended.

Note that in a case in which step S15 is executed again after step S19, when the determination unit 52 determines to execute the electric power consumption reducing processing or the wireless transmission amount reducing processing at a step to be described later and each parameter is set to a parameter used in the constraint processing and the recovery processing, step S15 is executed again after the parameter determination unit 53 of the parameter control device 5 sets each parameter back to a parameter used in the standard processing and the parameter transmission unit 54 of the parameter control device 5 executes processing that transmits each parameter.

As illustrated in FIG. 6, when the remaining amount of the battery 25A is smaller than the predetermined battery threshold value at step S16 in FIG. 5 or when the temperature of the grasping portion 2Ba is equal to or higher than the predetermined temperature threshold value at step S17 in FIG. 5, the determination unit 52 subsequently determines the information related to the endoscope scene (step S21). When the determination unit 52 determines that the endoscope scene is an important scene, in other words, the high-resolution scene (Yes), the determination unit 52 subsequently determines whether the electric power consumption reducing processing can be executed only by reducing the illumination light amount (step S22). This determination is performed based on the information related to the remaining amount of the battery 25A and the information related to the temperature of the grasping portion 2Ba. Specifically, the determination unit 52 determines that the electric power consumption reducing processing can be executed only by reducing the illumination light amount when the remaining amount of the battery 25A is smaller than the battery threshold value but close to the battery threshold value or when the temperature of the grasping portion 2Ba is higher than the temperature threshold value but close to the temperature threshold value and it is unlikely that the endoscope 2 anomalously stops or the user cannot grip the grasping portion 2Ba.

When the determination unit 52 determines that the electric power consumption reducing processing can be executed only by reducing the illumination light amount at step S22 (Yes), the illumination unit 22 subsequently reduces the illumination light amount (step S23). Subsequently, the filter processing unit 34A executes the strong filter processing in which the effect of the filter processing is significantly stronger, and the multiplication processing unit 34B executes the strong multiplication processing in which the effect of the multiplication processing is significantly stronger (step S24).

In the present embodiment, step S23 is achieved when the determination unit 52 determines to execute only the illumination light amount change processing as the electric power consumption reducing processing. Step S24 is achieved when the determination unit 52 determines to execute the strong filter processing having an effect significantly stronger than when the recovery processing is not executed, and execute the strong multiplication processing having an effect significantly stronger than when the recovery processing is not executed.

When the determination unit 52 does not determine that the electric power consumption reducing processing can be executed only by reducing the illumination light amount at step S22 (NO), the illumination unit 22 subsequently reduces the illumination light amount and the compression processing unit 23A executes the compression processing in which the compression ratio is slightly higher (step S25). Subsequently, the filter processing unit 34A executes the strong filter processing in which the effect of the filter processing is moderately stronger, and the multiplication processing unit 34B executes the strong multiplication processing in which the effect of the multiplication processing is significantly stronger (step S26).

In the present embodiment, step S25 is achieved when the determination unit 52 determines to execute both the illumination light amount change processing and the compression amount change processing so that the amount of reduction of electric power consumption of the battery 25A through the illumination light amount change processing is larger than the amount of reduction of electric power consumption of the battery 25A through the compression amount change processing. Step S26 is achieved when the determination unit 52 determines to execute the strong filter processing having an effect moderately stronger than when the recovery processing is not executed, and execute the strong multiplication processing having an effect significantly stronger than when the recovery processing is not executed.

When the determination unit 52 determines that the endoscope scene is not an important scene, in other words, not the high-resolution scene at step S21 (No), the illumination unit 22 subsequently significantly reduces the illumination light amount, and the compression processing unit 23A executes the compression processing in which the compression ratio is significantly higher (step S27). Subsequently, the filter processing unit 34A executes the strong filter processing in which the effect of the filter processing is slightly stronger, and the multiplication processing unit 34B executes the strong multiplication processing in which the effect of the multiplication processing is significantly stronger (step S28).

In the present embodiment, step S27 is achieved when the determination unit 52 determines to execute both the illumination light amount change processing and the compression amount change processing. Step S28 is achieved when the determination unit 52 determines to execute the strong filter processing having an effect slightly stronger than when the recovery processing is not executed, and execute the strong multiplication processing having an effect significantly stronger than when the recovery processing is not executed.

A setting example of the parameters for achieving steps S23 to S28 will be described later.

After step S24, S26, or S28 is executed, for example, the main control unit 36 determines whether to power off the endoscope system 1 (step S29). Contents of step S29 are the same as contents of step S19 in FIG. 5. When the main control unit 36 determines not to power off the endoscope system 1 (No), step S15 in FIG. 5 is executed again. When the main control unit 36 determines to power off the endoscope system 1 (Yes), the series of operations are ended.

As illustrated in FIG. 7, when the determination unit 52 determines that the wireless environment is degraded at step S18 in FIG. 5 (Yes), the determination unit 52 subsequently determines the information related to the endoscope scene (step S31). When the determination unit 52 determines that the endoscope scene is an important scene, in other words, the high-resolution scene (Yes), the determination unit 52 subsequently determines whether the wireless environment is slightly degraded (step S32). This determination is performed based on the information related to the wireless environment. Specifically, the determination unit 52 determines that the wireless environment is slightly degraded, for example, when the forwardable data amount is smaller than the predetermined threshold value but close to the predetermined threshold value.

When the determination unit 52 determines that the wireless environment is slightly degraded at step S32 (Yes), the compression processing unit 23A subsequently executes the compression processing in which the compression ratio is slightly higher (step S33). Subsequently, the filter processing unit 34A executes the weak filter processing in which the effect of the filter processing is slightly weaker, and the multiplication processing unit 34B executes the strong multiplication processing in which the effect of the multiplication processing is slightly stronger (step S34).

In the present embodiment, step S33 is achieved when the determination unit 52 determines to execute the compression amount change processing. Step S34 is achieved when the determination unit 52 determines to execute the weak filter processing having an effect slightly weaker than when the recovery processing is not executed, and execute the strong multiplication processing having an effect slightly stronger than when the recovery processing is not executed.

When the determination unit 52 determines that the wireless environment is not slightly degraded at step S32 (No), the compression processing unit 23A subsequently executes the compression processing in which the compression ratio is moderately higher (step S35). Subsequently, the filter processing unit 34A executes the weak filter processing in which the effect of the filter processing is moderately weaker, and the multiplication processing unit 34B executes the strong multiplication processing in which the effect of the multiplication processing is moderately stronger (step S36).

In the present embodiment, step S35 is achieved when the determination unit 52 determines to execute the compression amount change processing. Step S36 is achieved when the determination unit 52 determines to execute the weak filter processing having an effect moderately weaker than when the recovery processing is not executed, and execute the strong multiplication processing having an effect moderately stronger than when the recovery processing is not executed.

When the determination unit 52 determines that the endoscope scene is not an important scene, in other words, not the high-resolution scene at step S31 (No), the compression processing in which the compression ratio is significantly higher is subsequently executed (step S37). Subsequently, the filter processing unit 34A executes the weak filter processing in which the effect of the filter processing is significantly weaker, and the multiplication processing unit 34B executes the strong multiplication processing in which the effect of the multiplication processing is significantly stronger (step S38).

In the present embodiment, step S37 is achieved when the determination unit 52 determines to execute the compression amount change processing. Step S38 is achieved when the determination unit 52 determines to execute the weak filter processing having an effect significantly weaker than when the recovery processing is not executed, and execute the strong multiplication processing having an effect significantly stronger than when the recovery processing is not executed.

A setting example of the parameters for achieving steps S33 to S38 will be described later.

After step S34, S36, or S38 is executed, for example, the main control unit 36 determines whether to power off the endoscope system 1 (step S39). Contents of step S39 are the same as the contents of step S19 in FIG. 5. When the main control unit 36 determines not to power off the endoscope system 1 (No), step S15 in FIG. 5 is executed again. When the main control unit 36 determines to power off the endoscope system 1 (Yes), the series of operations are ended.

(Setting Example of Parameters)

Subsequently, a setting example of the parameters will be described below. In this example, the illumination parameter, the compression parameter, the first brightness parameter, and the second brightness parameter are each expressed by using a value of one to five inclusive. It is set that the illumination light amount is strongest when the value of the illumination parameter is one, and the illumination light amount is weakest when the value is five. In other words, it is set that an effect of the electric power consumption reducing processing is weakest when the value of the illumination parameter is one, and the effect of the electric power consumption reducing processing is strongest when the value is five.

It is set that the compression ratio is lowest when the value of the compression parameter is one, and the compression ratio is highest when the value is five. In other words, it is set that the effect of the electric power consumption reducing processing or an effect of the wireless transmission amount reducing processing is weakest when the value of the compression parameter is one, and the effect of the electric power consumption reducing processing or the effect of the wireless transmission amount reducing processing is strongest when the value is five.

It is set that the effect of the filter processing is weakest when the value of the first brightness parameter is one, and the effect of the filter processing is strongest when the value is five. It is set that the effect of the multiplication processing is weakest when the value of the second brightness parameter is one, and the effect of the multiplication processing is strongest when the value is five. Brightness of a correction target pixel is lowest when the effect of the filter processing or the multiplication processing is weakest, and is highest when the effect of the filter processing or the multiplication processing is strongest.

Hereinafter, default values are defined to be the values of the parameters when none of the constraint processing and the recovery processing is executed and the endoscope scene is the detailed-check scene. The default values are three. First, a setting example of the parameters when none of the constraint processing and the recovery processing is executed, in other words, when the standard processing is executed will be described with reference to Table 1. Table 1 presents the setting example of the parameters when the standard processing is executed and the endoscope scene is the detailed-check scene, the screening scene, and the external scene.

TABLE 1

| | Detailed- | | |
|---|---|---|---|
| | check | Screening | External |
| Parameter | scene | scene | scene |
| Illumination | 3 | 4 | 5 |
| parameter | | | |
| Compression | 3 | 4 | 5 |
| parameter | | | |
| First brightness | 3 | 4 | 5 |
| parameter | | | |
| Second brightness | 3 | 4 | 5 |
| parameter | | | |

The illumination parameter, the compression parameter, the first brightness parameter, and the second brightness parameter are set so that the image quality and the resolution of the endoscope image are highest when the standard processing is executed and the endoscope scene is the detailed-check scene. In the external scene, the image quality and the resolution of the endoscope image may be low. Thus, in the external scene, the illumination parameter and the compression parameter are set so that consumption of electric power of the battery 25A is smallest, and the first and second brightness parameters are set in accordance with the setting of the illumination parameter and the compression parameter. In the screening scene, the illumination parameter, the compression parameter, the first brightness parameter, and the second brightness parameter are set so that the image quality and the resolution of the endoscope image are higher than in the external scene but consumption of electric power of the battery 25A is smaller than in the detailed-check scene.

Subsequently, a setting example of the parameters when the electric power consumption reducing processing is executed will be described with reference to Table 2. Steps S23 to S28 illustrated in FIG. 6 are executed when the determination unit 52 determines to execute the electric power consumption reducing processing. Table 2 presents the setting example of the parameters in a case in which steps S23 and S24 are executed, a case in which steps S25 and S26 are executed, and a case in which steps S27 and S28 are executed.

TABLE 2

| Parameter | S23, S24 | S25, S26 | S27, S28 |
|---|---|---|---|
| Illumination | 3.5 | 3.5 | 4 |
| parameter | | | |
| Compression | 3 | 3.25 | 4 |
| parameter | | | |
| First brightness | 4 | 3.5 | 3.25 |
| parameter | | | |
| Second brightness | 4 | 4 | 4 |
| parameter | | | |

Steps S23 and S24 are executed when it is determined that the endoscope scene is the high-resolution scene and the electric power consumption reducing processing can be executed only by reducing the illumination light amount. In this case, the parameters are set so that the endoscope image of high image quality and high resolution can be obtained despite of the execution of the electric power consumption reducing processing. Specifically, the illumination parameter is set to a value (in Table 2, 3.5) with which the illumination light amount of the illumination unit 22 is smaller than when the electric power consumption reducing processing is not executed. The first brightness parameter is set to a value (in Table 2, 4) with which the effect of the filter processing is significantly stronger than when the recovery processing is not executed. The second brightness parameter is set to a value (in Table 2, 4) with which the effect of the multiplication processing is significantly stronger than when the recovery processing is not executed.

Steps S25 and S26 are executed when it is determined that the endoscope scene is the high-resolution scene and the electric power consumption reducing processing cannot be executed only by reducing the illumination light amount. In this case, the parameters are set so that the image quality and the resolution of the endoscope image are lower but the effect of the electric power consumption reducing processing is stronger than when steps S23 and S24 are executed. Specifically, the illumination parameter is set to a value (in Table 2, 3.5) with which the illumination light amount of the illumination unit 22 is smaller than when the electric power consumption reducing processing is not executed. The compression parameter is set to a value (in Table 2, 3.25) with which the data amount of the compressed data is slightly smaller than when the electric power consumption reducing processing is not executed. The first brightness parameter is set to a value (in Table 2, 3.5) with which the effect of the filter processing is moderately stronger than when the recovery processing is not executed. The second brightness parameter is set to a value (in Table 2, 4) with which the effect of the multiplication processing is significantly stronger than when the recovery processing is not executed.

Steps S27 and S28 are executed when the determination unit 52 determines that the endoscope scene is not the high-resolution scene. In this case, the parameters are set so that the endoscope image of minimum image quality and resolution with which, for example, the insertion portion 2A can be removed out of the body can be obtained despite of enhancement of the effect of the electric power consumption reducing processing. Specifically, the illumination parameter is set to a value (in Table 2, 4) with which the illumination light amount of the illumination unit 22 is significantly smaller than when the electric power consumption reducing processing is not executed. The compression parameter is set to a value (in Table 2, 4) with which the data amount of the compressed data is significantly smaller than when the electric power consumption reducing processing is not executed. The first brightness parameter is set to a value (in Table 2, 3.25) with which the effect of the filter processing is slightly stronger than when the recovery processing is not executed. The second brightness parameter is set to a value (in Table 2, 4) with which the effect of the multiplication processing is significantly stronger than when the recovery processing is not executed.

Subsequently, a setting example of the parameters when the wireless transmission amount reducing processing is executed will be described with reference to Table 3. Steps S33 to S38 illustrated in FIG. 7 are executed when the determination unit 52 determines to execute the wireless transmission amount reducing processing. Table 3 presents the setting example of the parameters in a case in which steps S33 and S34 are executed, a case in which steps S35 and S36 are executed, and a case in which steps S37 and S38 are executed.

TABLE 3

| Parameter | S33, S34 | S35, S36 | S37, S38 |
|---|---|---|---|
| Illumination | 3 | 3 | 3 |
| parameter | | | |
| Compression | 3.25 | 3.5 | 4 |
| parameter | | | |
| First brightness | 2.75 | 2.5 | 2 |
| parameter | | | |
| Second brightness | 3.25 | 3.5 | 4 |
| parameter | | | |

Steps S33 and S34 are executed when the determination unit 52 determines that the endoscope scene is the high-resolution scene and the wireless environment is slightly degraded. In this case, the parameters are set so that the endoscope image of high image quality and high resolution can be obtained despite of execution of the wireless transmission amount reducing processing. Specifically, the compression parameter is set to a value (in Table 3, 3.25) with which the data amount of the compressed data is slightly smaller than when the wireless transmission amount reducing processing is not executed. The first brightness parameter is set to a value (in Table 2, 2.75) with which the effect of the filter processing is slightly weaker than when the recovery processing is not executed. The second brightness parameter is set to a value (in Table 3, 3.25) with which the effect of the multiplication processing is slightly stronger than when the recovery processing is not executed.

Steps S35 and S36 are executed when the determination unit 52 determines that the endoscope scene is the high-resolution scene and the wireless environment is not slightly degraded. In this case, the parameters are set so that the image quality and the resolution of the endoscope image are lower but the effect of the wireless transmission amount reducing processing is stronger than when steps S33 and S34 are executed. Specifically, the compression parameter is set to a value (in Table 3, 3.5) with which the data amount of the compressed data is moderately smaller than when the wireless transmission amount reducing processing is not executed. The first brightness parameter is set to a value (in Table 3, 2.5) with which the effect of the filter processing is moderately weaker than when the recovery processing is not executed. The second brightness parameter is set to a value (in Table 3, 3.5) with which the effect of the multiplication processing is moderately stronger than when the recovery processing is not executed.

Steps S37 and S38 are executed when the determination unit 52 determines that the endoscope scene is not the high-resolution scene. In this case, the parameters are set so that the endoscope image of minimum image quality and resolution can be obtained despite of enhancement of the effect of the wireless transmission amount reducing processing. Specifically, the compression parameter is set to a value (in Table 3, 4) with which the data amount of the compressed data is significantly smaller than when the wireless transmission amount reducing processing is not executed. The first brightness parameter is set to a value (in Table 3, 2) with which the effect of the filter processing is significantly weaker than when the recovery processing is not executed. The second brightness parameter is set to a value (in Table 3, 4) with which the effect of the multiplication processing is significantly stronger than when the recovery processing is not executed.

(Operations and Effects)

Subsequently, operations and effects of the endoscope system 1 and the parameter control device 5 according to the present embodiment will be described. In the present embodiment, the determination unit 52 of the parameter control device 5 determines contents of the constraint processing by determining a plurality of pieces of information collected by the data collection unit 51, and determines contents of the recovery processing that operates at least one of the endoscope 2 or the video processor 3 to recover a function for displaying the endoscope image, which is degraded through the constraint processing, specifically, the image quality maintaining function that maintains the image quality of the endoscope image at a predetermined level or higher. The parameter determination unit 53 of the parameter control device 5 determines one or more parameters used in the constraint processing having contents determined by the determination unit 52, and one or more parameters used in the recovery processing having contents determined by the determination unit 52. According to the present embodiment, the image quality maintaining function can be recovered by causing the endoscope 2 and the video processor 3 to execute the constraint processing and the recovery processing by using the plurality of parameters determined by the parameter determination unit 53 as described above.

In the present embodiment, the information related to the endoscope scene is included in the plurality of pieces of information collected by the data collection unit 51. With this configuration, according to the present embodiment, contents of the constraint processing and the recovery processing can be changed for each endoscope scene, and as a result, the endoscope image of optimum image quality and image can be obtained for each endoscope scene. Thus, according to the present embodiment, it is possible to satisfy user's needs for the endoscope image of high resolution in the high-resolution scene even when the constraint processing is executed.

In the present embodiment, the electric power consumption reducing processing is executed as one piece of the constraint processing. The electric power consumption reducing processing includes at least the illumination light amount change processing among the illumination light amount change processing and the compression amount change processing. Comparison for the same effect of the electric power consumption reducing processing indicates that the illumination light amount change processing can typically reduce decrease of the resolution of the endoscope image as compared to the compression amount change processing. In the present embodiment, when the determination unit 52 determines that the execution condition of the electric power consumption reducing processing is satisfied and that the endoscope scene is the high-resolution scene, the determination unit 52 determines to preferentially execute the illumination light amount change processing among the illumination light amount change processing and the compression amount change processing. With this configuration, according to the present embodiment, it is possible to reduce decrease of the resolution of the endoscope image at execution of the electric power consumption reducing processing and satisfy user's needs for the endoscope image of high resolution in the high-resolution scene.

Note that the brightness of the endoscope image decreases when the illumination light amount change processing is executed to decrease the illumination light amount. In contrast, in the present embodiment, the first and second brightness parameters are determined so that the effect of the brightness correction processing is stronger than when the recovery processing is not executed. With this configuration, according to the present embodiment, it is possible to reduce decrease of the brightness of the endoscope image.

In the present embodiment, the wireless transmission amount reducing processing is executed as the other piece of the constraint processing. The wireless transmission amount reducing processing includes the compression amount change processing. Typically, the resolution of the endoscope image decreases as the compression ratio increases, in other words, the data amount of the compressed data decreases. In the present embodiment, the filter processing is performed by the filter processing unit 34A. Typically, the resolution of the endoscope image decreases as the effect of the filter processing becomes stronger.

In contrast, in the present embodiment, the determination unit 52 determines to execute the weak filter processing as the recovery processing when the determination unit 52 determines that the execution condition of the wireless transmission amount reducing processing is satisfied and that the endoscope scene is the high-resolution scene. With this configuration, according to the present embodiment, it is possible to reduce decrease of the resolution of the endoscope image at execution of the wireless transmission amount reducing processing. In the above-described case, the determination unit 52 determines to execute the strong multiplication processing as the recovery processing. With this configuration, according to the present embodiment, it is possible to reduce decrease of the effect of the brightness correction processing.

### Second Embodiment

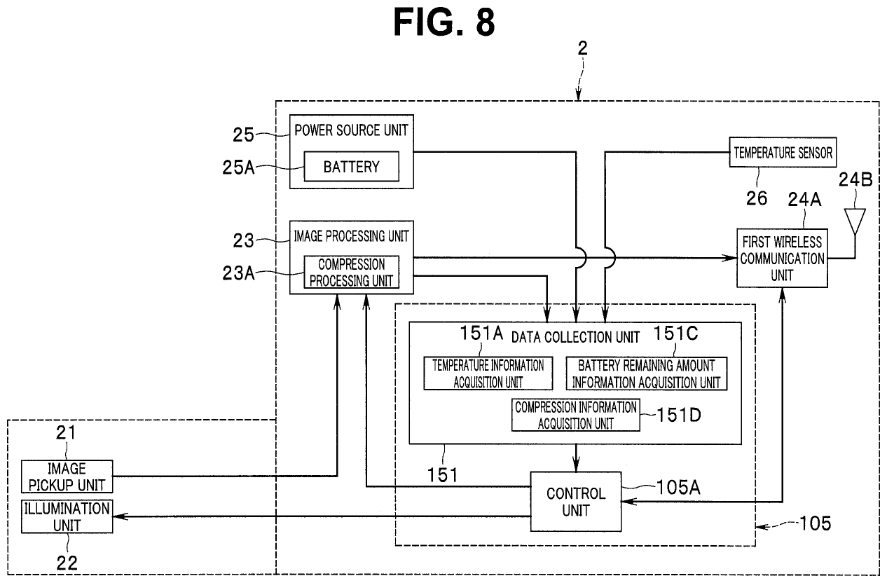

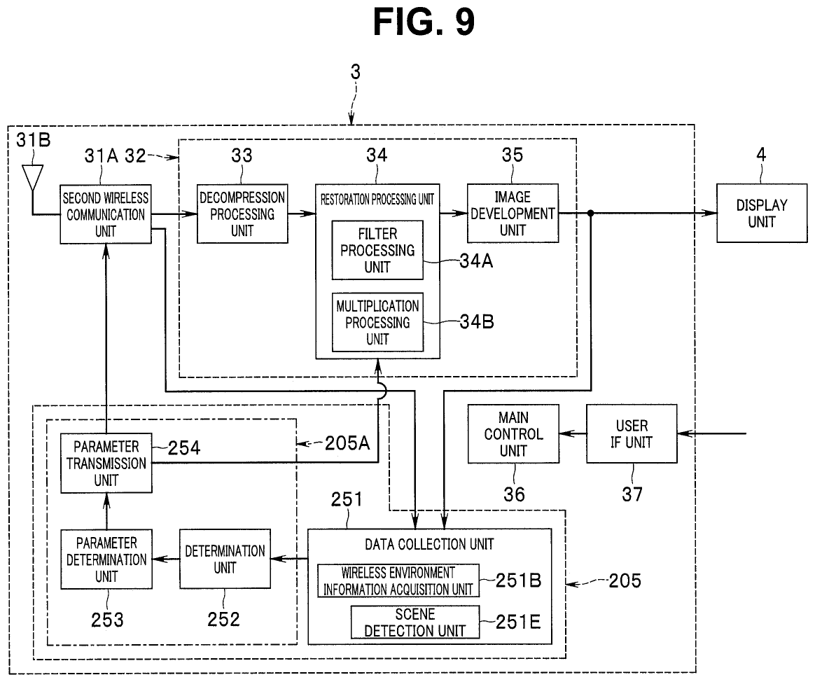

Subsequently, an endoscope system according to a second embodiment of the present invention will be described below with reference to FIGS. 8 and 9. FIG. 8 is a functional block diagram illustrating a configuration of an endoscope and a first part of a parameter control device in the endoscope system according to the present embodiment. FIG. 9 is a functional block diagram illustrating a configuration of a video processor and a second part of the parameter control device in the endoscope system according to the present embodiment. As illustrated in FIGS. 8 and 9, the endoscope system according to the present embodiment includes the parameter control device according to the present embodiment in place of the parameter control device 5 according to the first embodiment. The parameter control device according to the present embodiment includes a first part 105 provided in the endoscope 2, and a second part 205 provided in the video processor 3.

As illustrated in FIG. 8, the first part 105 of the parameter control device includes a data collection unit 151 and a control unit 105A. The data collection unit 151 includes a temperature information acquisition unit 151A, a battery remaining amount information acquisition unit 151C, and a compression information acquisition unit 151D. In other words, the temperature information acquisition unit 151A and the battery remaining amount information acquisition unit 151C are provided in the endoscope 2. Functions of the temperature information acquisition unit 151A, the battery remaining amount information acquisition unit 151C, and the compression information acquisition unit 151D are the same as functions of the temperature information acquisition unit 51A, the battery remaining amount information acquisition unit 51C, and the compression information acquisition unit 51D, respectively, in the first embodiment.

The data collection unit 151 outputs, to the control unit 105A, information related to the temperature of the grasping portion 2Ba, which is acquired by the temperature information acquisition unit 151A, information related to the remaining amount of the battery 25A, which is acquired by the battery remaining amount information acquisition unit 151C, and information related to the compression processing, which is acquired by the compression information acquisition unit 151D. The control unit 105A outputs the plurality of pieces of information acquired by the data collection unit 151 to the second part 205 of the parameter control device through wireless communication between the endoscope 2 and the video processor 3.

As illustrated in FIG. 9, the second part 205 of the parameter control device includes a data collection unit 251, a determination unit 252, a parameter determination unit 253, and a parameter transmission unit 254. The determination unit 252, the parameter determination unit 253, and the parameter transmission unit 254 are included in a control unit 205A as a main part of the parameter control device. In other words, the determination unit 252 and the parameter determination unit 253 are provided in the video processor 3.

The data collection unit 251 includes a wireless environment information acquisition unit 251B and a scene detection unit 251E. In other words, the wireless environment information acquisition unit 251B and the scene detection unit 251E are provided in the video processor 3.

Functions of the wireless environment information acquisition unit 251B are basically the same as functions of the wireless environment information acquisition unit 51B in the first embodiment. Note that, in the present embodiment, the second wireless communication unit 31A includes a non-illustrated environment detection circuit configured to detect the state of the wireless environment. The wireless environment information acquisition unit 251B acquires, as the information related to the wireless environment, a result of the detection by the environment detection circuit of the second wireless communication unit 31A or a forwardable data amount calculated based on the result of the detection by the environment detection circuit. Note that, in the present embodiment, the first wireless communication unit 24A may or may not include an environment detection circuit. In the former case, the first wireless communication unit 24A outputs information related to the wireless environment, which is detected by the environment detection circuit to the second part 205 of the parameter control device through wireless communication between the endoscope 2 and the video processor 3.

Functions of the scene detection unit 251E are basically the same as functions of the scene detection unit 51E in the first embodiment. Note that, in the present embodiment, the image processing unit 32 outputs, as information related to an endoscope scene, image data for detecting an endoscope scene to the second part 205 of the parameter control device. In an example illustrated in FIG. 9, the scene detection unit 251E receives the endoscope image outputted from the image development unit 35 of the image processing unit 32. The scene detection unit 251E detects an endoscope scene by analyzing acquired image data, in other words, the endoscope image.

The data collection unit 251 receives a plurality of pieces of data collected by the data collection unit 151 and outputted from the control unit 105A. Accordingly, the data collection unit 251 acquires the plurality of pieces of information acquired by the data collection unit 151 in effect.

The determination unit 252 determines contents of the constraint processing and contents of the recovery processing by determining the plurality of pieces of information acquired by the data collection unit 251 (including the plurality of pieces of information acquired by the data collection unit 151). A method of determining contents of the constraint processing and contents of the recovery processing is the same as in the first embodiment.

The parameter determination unit 253 determines one or more parameters used in the constraint processing having contents determined by the determination unit 252, and one or more parameters used in the recovery processing having contents determined by the determination unit 252. A method of the parameter determination is the same as in the first embodiment.

The parameter transmission unit 254 transmits the plurality of parameters determined by the parameter determination unit 253 to components of the endoscope 2 and the video processor 3. Specifically, the parameter transmission unit 254 transmits an illumination parameter and a compression parameter to the control unit 105A, transmits a first brightness parameter to the filter processing unit 34A of the restoration processing unit 34, and transmits a second brightness parameter to the multiplication processing unit 34B of the restoration processing unit 34. The control unit 105A outputs the received illumination parameter to the illumination unit 22, and outputs the received compression parameter to the compression processing unit 23A.

In the present embodiment, the control unit 205A as the main part of the parameter control device is provided in the video processor 3. With this configuration, according to the present embodiment, consumption of electric power of the battery 25A can be reduced as compared to a configuration in which the main part of the parameter control device is provided in the endoscope 2.

Other configurations, operations, and effects in the present embodiment are the same as the configurations, the operations, and the effects in the first embodiment.

The present invention is not limited to the above-described embodiments but may be provided with various kinds of changes, modifications, and the like without changing the gist of the present invention. For example, each parameter control device of the present invention may be a device separated from the endoscope 2 and the video processor 3.

The wireless environment information acquisition unit and the scene detection unit of each data collection unit may be provided in both the endoscope 2 and the video processor 3.

1. A video processor comprising a processor, wherein
the processor is configured to:
acquire at least one piece of information of information related to temperature of a grasping portion of an endoscope, information related to wireless environment of wireless communication that transmits and receives image data obtained through image pickup by the endoscope, or information related to a remaining amount of a battery of the endoscope; and
control a plurality of parameters, and
the processor:
determines, based on the at least one piece of information, contents of constraint processing that selectively constrains operation of the endoscope and contents of recovery processing that recovers a function for displaying an endoscope image, the function being degraded through the constraint processing; and
determines a parameter for the constraint processing and a parameter used in the recovery processing.

2. The video processor according to claim 1, wherein the processor is further configured to acquire information related to an endoscope scene.

3. The video processor according to claim 2, wherein
when the processor determines at least one of a condition that the temperature of the grasping portion is equal to or higher than a predetermined temperature threshold value or a condition that the remaining amount of the battery is smaller than a predetermined battery threshold value, the processor determines to execute electric power consumption reducing processing as the constraint processing,
the electric power consumption reducing processing includes at least illumination light amount change processing among the illumination light amount change processing and compression amount change processing, the illumination light amount change processing being processing that changes an illumination light amount of an illumination element configured to illuminate an object, the compression amount change processing being processing that changes a data amount of compressed data generated by compressing the image data,
when the processor determines to execute the illumination light amount change processing, the processor determines an illumination parameter so that the illumination light amount is smaller than when the electric power consumption reducing processing is not executed, the illumination parameter defining the illumination light amount, and
when the processor determines to execute the compression amount change processing, the processor determines a compression parameter so that the data amount of the compressed data is smaller than when the electric power consumption reducing processing is not executed, the compression parameter defining the data amount of the compressed data.

4. The video processor according to claim 3, wherein when the processor determines at least one of the condition that the temperature of the grasping portion is equal to or higher than the predetermined temperature threshold value or the condition that the remaining amount of the battery is smaller than the predetermined battery threshold value, and determines that the endoscope scene is a scene that needs the endoscope image of high resolution, the processor determines to preferentially execute the illumination light amount change processing among the illumination light amount change processing and the compression amount change processing.

5. The video processor according to claim 4, wherein the processor determines to execute only the illumination light amount change processing.

6. The video processor according to claim 4, wherein the processor determines the illumination parameter and the compression parameter so that an amount of reduction of electric power consumption of the battery through the illumination light amount change processing is larger than an amount of reduction of electric power consumption of the battery through the compression amount change processing.

7. The video processor according to claim 3, wherein
the processor is further configured to perform predetermined image processing on the image data,
the image processing includes brightness correction processing that corrects brightness of decompressed image data generated by decompressing the compressed data, and
when the processor determines to execute the electric power consumption reducing processing, the processor determines, as the recovery processing, to change contents of the brightness correction processing and determines a brightness parameter so that an effect of the brightness correction processing that brightens the endoscope image is stronger than when the recovery processing is not executed, the brightness parameter defining a relation between brightness of the decompressed image data before correction and brightness of the decompressed image data after correction.

8. The video processor according to claim 7, wherein when the processor determines that the endoscope scene is a scene that needs the endoscope image of high resolution, and determines to change the contents of the brightness correction processing, the processor determines the brightness parameter so that the effect of the brightness correction processing is stronger than when the recovery processing is not executed but the effect of the brightness correction processing is weaker than when it is not determined that the endoscope scene is a scene that needs the endoscope image of high resolution.

9. The video processor according to claim 1, wherein
when the processor determines that a forwardable data amount of the wireless communication is smaller than a predetermined threshold value, the processor determines to execute wireless transmission amount reducing processing as the constraint processing,
the wireless transmission amount reducing processing includes compression amount change processing that changes a data amount of compressed data generated by compressing the image data, and
when the processor determines to execute the wireless transmission amount reducing processing, the processor determines a compression parameter so that the data amount of the compressed data is smaller than when the wireless transmission amount reducing processing is not executed, the compression parameter defining the data amount of the compressed data.

10. The video processor according to claim 9, wherein
the processor is further configured to acquire information related to an endoscope scene, and
when the processor determines that the endoscope scene is a scene that needs the endoscope image of high resolution, and determines to execute the wireless transmission amount reducing processing, the processor determines the compression parameter so that the data amount of the compressed data is smaller than when the wireless transmission amount reducing processing is not executed but the data amount of the compressed data is larger than when it is not determined that the endoscope scene is a scene that needs the endoscope image of high resolution.

11. The video processor according to claim 9, wherein
the processor is further configured to perform predetermined image processing on the image data,
the image processing includes brightness correction processing that corrects brightness of decompressed image data generated by decompressing the compressed data,
the brightness correction processing includes filter processing and multiplication processing, the filter processing being processing that corrects brightness of any one pixel of the decompressed image data by using a plurality of pixel values in a predetermined region including the one pixel and a plurality of pixels surrounding the one pixel and a first brightness parameter, the multiplication processing being processing that corrects the brightness of the one pixel by using a pixel value of the one pixel and a second brightness parameter, and
when the processor determines to execute the wireless transmission amount reducing processing, the processor determines, as the recovery processing, to execute weak filter processing and strong multiplication processing, determines the first brightness parameter so that an effect of the filter processing is weaker than when the recovery processing is not executed, and determines the second brightness parameter so that an effect of the multiplication processing is stronger than when the recovery processing is not executed, the weak filter processing being the filter processing having an effect weaker than when the recovery processing is not executed, the strong multiplication processing being the multiplication processing having an effect stronger than when the recovery processing is not executed.

12. The video processor according to claim 11, wherein
the processor is further configured to acquire information related to an endoscope scene, and
when the processor determines that the endoscope scene is a scene that needs the endoscope image of high resolution, and determines to execute the weak filter processing and the strong multiplication processing, the processor determines the first brightness parameter so that the effect of the filter processing is weaker than when the recovery processing is not executed but the effect of the filter processing is stronger than when it is not determined that the endoscope scene is a scene that needs the endoscope image of high resolution, and determines the second brightness parameter so that the effect of the multiplication processing is stronger than when the recovery processing is not executed but the effect of the multiplication processing is weaker than when it is not determined that the endoscope scene is a scene that needs the endoscope image of high resolution.

13. The video processor according to claim 2, wherein
the processor is further configured to:
acquire the information related to the temperature of the grasping portion;
acquire the information related to the wireless environment;
acquire the information related to the remaining amount of the battery; and
acquire the information related to the endoscope scene,
the acquisition of the information related to the temperature of the grasping portion and the acquisition of the information related to the remaining amount of the battery are executed by the endoscope, and
the acquisition of the information related to the wireless environment and the acquisition of the information related to the endoscope scene are executed by at least one of the endoscope or the video processor.

14. The video processor according to claim 13, wherein the acquisition of the information related to the wireless environment and the acquisition of the information related to the endoscope scene are executed by the endoscope.

15. The video processor according to claim 13, wherein the acquisition of the information related to the wireless environment and the acquisition of the information related the endoscope scene are executed by the video processor.

16. An image processing method of generating an endoscope image from image data acquired by an image pickup device of an endoscope, the image processing method comprising:
acquiring at least one piece of information of information related to temperature of a grasping portion of the endoscope, information related to wireless environment of wireless communication that transmits and receives the image data, or information related to a remaining amount of a battery of the endoscope;
determining, based on the at least one piece of information, contents of constraint processing that selectively constrains operation of the endoscope and contents of recovery processing that recovers a function for displaying the endoscope image, the function being degraded through the constraint processing; and
determining a parameter for the constraint processing and a parameter used in the recovery processing.

17. An endoscope comprising a processor, wherein
the processor is configured to:
acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and
control a plurality of parameters, and
the processor:
determines, based on the at least one piece of information, contents of constraint processing that selectively constrains operation of the endoscope and contents of recovery processing that recovers a function for displaying an endoscope image, the function being degraded through the constraint processing; and
determines a parameter for the constraint processing and a parameter used in the recovery processing.

18. An endoscope system comprising:
an endoscope;
a video processor; and
a processor,
wherein the processor is configured to:
acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and
control a plurality of parameters, and
the processor:
determines, based on the at least one piece of information, contents of constraint processing that selectively constrains operation of the endoscope and contents of recovery processing that recovers a function for displaying an endoscope image, the function being degraded through the constraint processing; and
determines a parameter for the constraint processing and a parameter used in the recovery processing, and
the processor is provided in the endoscope.

19. An endoscope system comprising:
an endoscope;
a video processor; and
a processor,
wherein the processor is configured to:
acquire at least one piece of information of information related to temperature of a grasping portion of the endoscope, information related to wireless environment of wireless communication that transmits and receives image data obtained through image pickup by the endoscope, or information related to a remaining amount of a battery of the endoscope; and
control a plurality of parameters, and
the processor:
determines, based on the at least one piece of information, contents of constraint processing that selectively constrains operation of the endoscope and contents of recovery processing that recovers a function for displaying an endoscope image, the function being degraded through the constraint processing; and
determines a parameter for the constraint processing and a parameter used in the recovery processing, and
the processor is provided in the video processor.
